(19) United States
(12) Patent Application Publication
(10) Pub. No.: US 2010/0291249 A1
(43) Pub. Date: Nov. 18, 2010

(54) Pomegranate-derived Products for the Treatment of Skin Sores and Lesions

(75) Inventor: Mordechai Klein, Ramat Gan (IL)

Correspondence Address: MDIP LLC, POST OFFICE BOX 2630, MONTGOMERY VILLAGE, MD, 20886-2630, US

(73) Assignee: ORI SOFTWARE, Ashkelon (IL)

(21) Appl. No.: 12/676,957

(22) PCT Filed: Sep. 7, 2008
(86) PCT No.: PCT/IL08/01203

§ 371 Date: Jul. 27, 2010

(30) Foreign Application Priority Data
Sep. 6, 2007 (IL) 185775

(51) Int. Cl. A61K 36/185; A61P 17/00; A61P 17/10; A61P 17/02; C12P 1/00

(52) U.S. Cl. 424/776; 424/778; 435/267

(57) ABSTRACT

A method of treating skin sores is described, using medical compositions derived from pomegranate fruit. Methods for preparing such medical compositions are also described, including pomegranate seed oil extract, pomegranate inflorescence and peel extracts, and pomegranate peel powder.

### FIELD OF THE INVENTION

The present invention relates to pomegranate (Punica granatum) products, methods for their preparation and use thereof in treating skin sores and lesions, particularly skin sores associated with Type II diabetes.

### BACKGROUND OF THE INVENTION

One major property of pomegranate hulls is their strong astringency, making them a popular remedy throughout the world, in the form of an aqueous decoction (i.e., boiling the hulls in water for 10-40 minutes), for dysentery and diarrhea, and also for stomatitis. The decoction can be drunk, or used as a mouthwash, douche or enema.

Other ethno-medical explorations have documented pomegranate hull extract usage both orally and intravaginally to prevent fertility and abortion, and to ameliorate various gynecological problems. Other traditional uses of these materials have included treatments for snakebite, diabetes (Singh, 1986), burns and leprosy.

It is well known that the dried pomegranate seeds contain an oil, which has been shown to contain not only the steroidal estrogen estrone, but also the isoflavonic phytoestrogens genistein and daidzein, and the phytoestrogenic coumesten, coumestrol. Both the juice and the oil contain numerous and diverse bioflavonoids, which have been shown to be both potently antioxidant and inhibitory of one or both of the enzymes cyclooxygenase (catalyzing arachidonic acid to prostaglandins) and lipoxygenase (catalyzing arachidonic acid to leukotrienes). Extracts of the rinds have been shown to be bactericidal, antiviral and antitumor. When injected, a hydrolyzed concentrate of the oil produced increased uterine weight in immature rabbits, and cornification of vaginal epithelium in ovariectomized adult mice, both indicative of estrogenic action in vivo. Furthermore, recent studies have proven the simple extracts of the hulls to have efficacy against a broad range of bacteria, parasites and viruses.

Pomegranate-preparations components are in commercial use as ingredients of dietary and other products and are safe to use.

US 2002/0012710 discloses a mixture of a pomegranate seed oil product and a pomegranate juice product and a pharmaceutical composition containing the same. US 2002/0012710 also discloses a selective estrogen receptor modulator and other biologically active compounds derived from pomegranates as well as methods of use thereof.

U.S. Pat. No. 6,387,418 discloses an antioxidative composition comprising an extract from pomegranate, a method of reducing lipid peroxidation, aggregation or retention, HDL oxidation in a sample and a method of alleviating atherosclerosis in a patient.

WO 2005/070412 discloses administering orally or parentally a therapeutically effective amount of punicic acid (commonly found in pomegranate) to an animal, for enhancing the immune response of said animal, to prevent or ameliorate immunoinflammatory diseases, increase immune system development, increase immune function, increase immune response against viruses and prevent or ameliorate Metabolic Syndrome, Type 2 diabetes and obesity.

It is known that one of the main constituents of pomegranate seed oil, linolenic acid, has been correlated with lowering blood sugar in diabetic animals. Additionally, the systemic use of pomegranate seed oil in the treatment of diabetes is well known. The use of pomegranate seed oil for skin treatment is also familiar.

Diabetics may experience many serious, long-term complications, which may begin even within months of the onset of the disease, although most tend to develop after a few years. One of the most common complications is the “diabetic foot”, and diabetic sores. High sugar levels cause narrowing of both the small and large blood vessels, which therefore supply less blood, especially to the skin and nerves. The poor circulation to the skin can lead to ulcers and infections, and all wounds heal slowly. Moreover, ulcers and infections of the feet and legs are likely to develop. People with diabetes often develop bacterial and fungal infections, typically of the skin, and any infection that develops tends to be more severe. Too often, these wounds heal slowly or not at all, and amputation of the foot or part of the leg may be needed.

There exists therefore a need for effective treatment of diabetic sores and diabetic foot.

It is an object of the present invention to provide such pomegranate derived compositions effective in the treatment of diabetic sores and other sores, such as pressure sores, ulcers and acne. It is another object of the invention to provide methods for the preparation of pomegranate seed oil extracts, pomegranate flower and peel extracts and pomegranate peel powder, suitable for manufacturing a product useful for treating skin sores, particularly pressure sores and diabetes sores. It is another object of the present invention to provide a kit for the treatment of skin sores and lesions, particularly diabetic and pressure sores.

The present invention provides for these and more.

### SUMMARY OF THE INVENTION

In a first aspect, the present invention provides a process for the preparation of pomegranate seeds oil extract comprising the following steps:

a) providing isolated pomegranate seeds;
b) cold-pressing the seeds to obtain an oil; and optionally
c) pressure-filtering the resulting oil, to obtain clear oil.

In one embodiment, said isolated pomegranate seeds are isolated from the fermentation product of pomegranate sacs fermented with natural yeast.

More specifically, the invention relates to a process for the preparation of pomegranate seeds oil extract from pomegranates, comprising the following steps: a) separating the pomegranate sacs from the peel; b) squeezing the sacs to obtain pomegranate juice; c) introducing the sacs into a fermentation vessel together with natural yeast, and fermenting at a temperature suitable for yeast fermentation, preferably between 20° C. and 35° C., while mixing at suitable intervals; d) washing the resulting material with clean water while constantly vortexing; e) drying the clean seeds at 45° C. for a suitable period of time; f) transferring the dry seeds to a vibrator for the removal of any remaining pomegranate peel; g) separating the dry and clean seeds from the dry and clean pomegranate peel; h) cold-pressing the dry seeds to obtain oil; and optionally i) pressure-filtering the resulting oil, to obtain clear oil.

In another aspect, the invention relates to a process for the preparation of pomegranate-flower extract, comprising the following steps: a) immersing pomegranate inflorescence in 70% alcohol at room temperature for a suitable period of time, while mixing at suitable intervals; b) steaming and thickening the mixture obtained in step (a) until the alcohol has been removed to give a pomegranate flower extract.

The invention also relates to a process for the preparation of pomegranate peel extract from pomegranates, comprising the following steps: a) immersing pomegranate inflorescence in 70% alcohol at room temperature for a suitable period of time, while mixing at suitable intervals; b) steaming and thickening the mixture obtained in step (a) until the alcohol has been removed to give a pomegranate peel extract.

In accordance with the invention, a pomegranate inflorescence/peel extract can be prepared, comprising mixing the inflorescence extract obtained by the above process with the pomegranate peel extract obtained by the above process at a 1:1 ratio (v/v).

Still further, the invention relates to a process for the preparation of pomegranate peel powder from pomegranates, comprising the steps of: a) crushing whole pomegranate using a grinder; b) transferring the dry material (i.e. peel and seeds) into a fermentation vessel together with natural yeast and fermenting at a temperature suitable for yeast fermentation, preferably between 20° C. and 35° C., while mixing at suitable intervals; c) washing the resulting material with clean water to remove the seed membrane; d) drying the material obtained in step (c) in a drying room at 42° C.; e) transferring the dry material obtained in step (d) to a vibrator for the separation of the peel from the seeds; and e) grinding the dry peel to powder.

In all the processes described herein, optimal yeast fermentation temperature is between 25° C. and 30° C. The most preferred fermentation temperature is 28° C.

The invention also relates to pomegranate seed oil extract, A pomegranate flower extract, pomegranate peel extract, pomegranate inflorescence/peel extract and pomegranate peel powder, obtainable by the processes of the invention.

In yet another aspect, the invention relates to a medical or cosmetic composition for the treatment of skin sores, comprising as active ingredient at least one of pomegranate seed oil and pomegranate flower/peel extract. The pomegranate seed oil extract, and/or the pomegranate flower+peel extract and/or said pomegranate peel powder comprised in the compositions of the invention may be obtained by the respective process of the invention.

The compositions of the invention are particularly suitable for the treatment of diabetic sores, pressure sores, ulcers or sores and pimples typical of acne vulgaris.

The invention also relates to a kit for the treatment of skin sores comprising a) pomegranate flower+peel extract, preferably contained in an atomizer; b) pomegranate oil extract; c) pomegranate peel powder; d) means for applying the said pomegranate flower+peel extract, pomegranate oil extract and/or pomegranate peel powder to said skin sores; e) optionally bandages; and f) instructions for use.

In the kit of the invention, the said pomegranate seed oil extract, pomegranate flower+peel extract and/or pomegranate peel powder are obtainable by the respective processes of the invention.

The kit of the invention is particularly suitable for the treatment of diabetic sores, pressure sores, ulcers or acne vulgaris.

The invention also relates to a method of treating skin sores, comprising the following steps: a) washing the sore thoroughly with clean water, and/or with dilute hydrogen peroxide solution, and drying it and its surroundings with absorption wipers; b) applying to the sore, preferably by spraying, pomegranate flowers+peel extract on the sore; c) applying to the sores, preferably by spraying, pomegranate oil extract; d) powdering with pomegranate peel powder; e) optionally bandaging the sore, particularly if in the sole; and f) repeating steps (a)-(d) at least twice a day.

Also in the method of treatment of the invention the said pomegranate seed oil extract and/or pomegranate flower+peel extract and/or pomegranate peel powder are obtainable by the respective processes of the invention.

The method of treatment of the invention is particularly suitable for the treatment of diabetic sores, pressure sores, ulcers or acne vulgaris.

The invention also relates to a method of preventing skin sores in a patient in need, comprising the following steps: a) applying to the skin of said patient, preferably by spraying, pomegranate oil extract; b) powdering with pomegranate peel powder; and optionally c) repeating steps (a)-(d) twice a day.

### BRIEF DESCRIPTION OF THE FIGURES

FIGS. 1a-1b:

FIG. 1a is a photograph of a foot sore of a diabetic patient (Case 1) before treatment.

FIG. 1b is a photograph of the foot shown in FIG. 1a, after 30 days of treatment.

FIGS. 2a-2d:

FIG. 2a is a photograph of a foot sore of a diabetic patient (Case 2) at the start of the treatment.

FIG. 2b is a photograph of the foot shown in FIG. 2a, during the treatment.

FIG. 2c is a photograph of the foot shown in FIGS. 2a and 2b at the end of the treatment.

FIG. 2d is a close-up of the photograph presented in FIG. 2c.

FIG. 3a-3e:

FIG. 3a is a photograph of a foot sore of a diabetic patient (Case 3) at the start of the treatment.

FIGS. 3b, 3c and 3d are photographs of the foot shown in FIG. 3a, at different stages during the treatment.

FIG. 3e is a photograph of the foot shown in FIGS. 3a-3d at the end of the treatment.

### DESCRIPTION OF THE INVENTION

In search for efficient agents for the treatment of diabetic sores and diabetic foot, the inventors developed pomegranate seed oil extract, pomegranate flower and peel extracts and pomegranate peel powder, and various mixtures and combinations thereof, which may be used in the treatment of skin sores, particularly diabetic sores and pressure sores, a kit comprising said products for the treatment of skin sores, particularly diabetic sores and pressure sores with specific instructions for achieving recovery from said sores, will be detailed below.

Thus, in a first aspect, the invention relates to compositions, specifically medical compositions, for the treatment of skin sores. The invention provides novel approaches for the preparation of different pomegranate products, such as pomegranate seed oil, pomegranate peel extract, pomegranate peel powder and pomegranate flowers extract, for use as active components of the compositions of the invention.

The medical composition of the invention may optionally further comprise, one or more carriers, excipients and/or additives as known in the art, or further optionally additional active agents.

The invention further relates to the use of said pomegranate products and suitable mixtures and combinations thereof in the manufacture of medicaments for treating skin sores, more particularly diabetic sores and diabetic foot, as well as pressure sores, ulcers and acne. The term skin sore as used herein includes, but is not limited to any skin lesion, diabetic sore, pressure sore, ulcer, blister, wound, and the like, as well as acne vulgaris.

The medical compositions of the invention are preferably for topical administration, and therefore may be formulated as transdermal patches, ointments, lotions, creams, gels, drops, suppositories, sprays, liquids and powders. Conventional pharmaceutical carriers, aqueous, powder or oily bases, thickeners and the like may be necessary or desirable.

The preparation of medical compositions is well known in the art and has been described in many articles and textbooks, see e.g., Remington's Pharmaceutical Sciences, Gennaro A. R. ed., Mack Publishing Co., Easton, Pa., 1990, and especially pp. 1521-1712 therein.

The invention further provides methods and protocols for the treatment of skin sores, more particularly diabetic sores and diabetic foot, pressure sores and ulcers, as well as acne vulgaris, employing the products of the invention. Moreover, the invention provides methods for preventing skin sores in individuals prone to developing skin sores, such as diabetics and individuals prone to pressure sores in subjects in need, for example hemi- or paraplegic patients, by applying the products of the invention to the skin or skin areas of the patient.

The compositions of the invention, the pomegranate seed oil, the pomegranate flower (inflorescence) extract, the pomegranate peel extract, and the pomegranate peel powder may be used alone or in combination, according to the specific protocol of treatment chosen by the medical practitioner in charge of the treatment. When used in combination, the compositions may be used concomitantly (at the same time), or sequentially (one after the other). When used sequentially, there may be a lag of time between the use of one composition and the other.

During the treatment, as described in the examples, the composition may be applied and then bandaged or not. By bandaged it is meant that after applying a composition of the invention to the wound, or a combination thereof, the wound is covered with a dressing. For the purposes of the present application, the terms “bandage” and “dressing” are used interchangeably.

Types of dressings suitable for the present invention include gauzes (which may be impregnated with an agent designed to help sterility or to speed healing), films, gels, foams, hydrocolloids, alginates, hydrogels and polysaccharide pastes, granules and beads. Many gauze dressings have a layer of nonstick film over the absorbent gauze to prevent the wound from adhering to the dressing. Dressings may also be impregnated with antiseptic chemicals.

In addition, occlusive dressings, made from substances impervious to moisture such as plastic or latex, may also be used to increase the rate of absorption of the topical medications described in the invention into the skin.

The “ideal” wound dressing is one that is sterile, breathable, and encourages a moist healing environment. This will then reduce the risk of infection, help the wound heal more quickly, and reduce scarring.

As used herein, “medical composition” is a synonym of “pharmaceutical composition”, and both mean therapeutically effective amounts of the compounds of the present invention, together with suitable diluents, preservatives, solubilizers, emulsifiers, adjuvant and/or carriers. A “therapeutically effective amount” as used herein refers to that amount which provides a therapeutic effect for a given condition, any skin sore, and administration regimen.

Further, as used herein “pharmaceutically acceptable carriers” are well known to those skilled in the art and include, but are not limited to, 0.01-0.1 M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil. Aqueous carriers include water, alcoholic/aqueous solutions, emulsions or suspensions, including saline and buffered media.

Thus, the pharmaceutical compositions of the invention may be prepared and applied as solutions, suspensions, or emulsions in a physiologically acceptable diluent with or without a pharmaceutical carrier.

The term “treating” as used herein refers to abrogating, inhibiting, slowing or reversing the progression of a skin sore, ameliorating clinical symptoms of a skin sore or preventing the appearance of clinical symptoms of a skin sore. The term “preventing” is defined herein as barring a subject from acquiring a disorder or disease, i.e. a skin sore, in the first place.

The term “administering” as used herein refers to a method for bringing a pomegranate-derived medical composition of the present invention and a skin sore (the wound) together in such a manner that the composition can affect the sore directly. In one embodiment, the present invention encompasses administering the compositions of the present invention to a subject in need.

The term “contacting” as used herein refers to bringing into contact the skin sore, or the wound, and the compositions described herein, under in vivo conditions.

The term “therapeutically effective amount” refers to the amount of a compound being administered which relieves to some extent one or more of the symptoms of the disorder being treated. Therapeutic effective doses for the pomegranate-derived compositions described herein can be estimated initially from the examples described herein or an animal model. A dose can be formulated in an animal model, and this dose can be used to more precisely determine useful doses in humans.

It was surprisingly found by the inventors that by using the pomegranate seed oil and the pomegranate peel powder in the treatment of diabetic sores and diabetic foot, a significant improvement in the sores was achieved, and the sores were practically cured.

In a further aspect, the present invention also relates to a process for the preparation of pomegranate seeds oil extract, comprising the following steps:

a) separating the pomegranate sacs from the peel;
b) squeezing the sacs under pressure to give pomegranate juice;
c) transferring the sacs into a fermentation vessel together with natural yeast, incubating for a suitable period of time, while mixing at suitable intervals;
d) washing the resulting seeds with clean water while constantly vortexing;
e) drying the clean seeds in a drying room;
f) transferring the dry seeds to a vibrator to remove any remaining pomegranate peel;
g) separating the dry and clean seeds from any pomegranate peel (the peel may be used for preparing pomegranate peel powder);
h) pressing the dry seeds to obtain pomegranate seed oil; and optionally
i) pressure-filtering the oil obtained in step (h), to give clear oil.

The clear oil can be used for preparation of various pharmaceutical and medical products and also cosmetic products. For example, it can be packed in capsules, preferably pierceable capsules, for topical application.

Surprisingly, it was found that the methods for producing pomegranate seeds oil extract described in the examples below are advantageous over the known methods, and that the pomegranate seeds oil extract produced thereby presents excellent therapeutic properties for treating skin sores.

The invention is also directed to methods of producing pomegranate flower and peel extracts and pomegranate peel powder.

Finally, the present invention also provides the use of any one of the pomegranate-derived products, such as the seed oil extract, the inflorescence/flower extract, peel extract, and peel powder, in the preparation of a composition for the treatment of skin sores in the subject in need.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Example 1

### Preparation of Pomegranate Flower and Peel Extract

1. Soak pomegranate inflorescence or pomegranate peel in alcohol for a suitable period of time, at room temperature, while mixing at suitable intervals.
2. Remove all alcohol by steam distillation to yield liquid flower or peel extracts, respectively.
3. Mix the obtained flower extract and the peel extract with each other at a 1:1 ratio (v/v), to give a liquid flower/peel extract.

The obtained liquid pomegranate flower and peel extract (flower+peel extract) possesses disinfecting and also curing properties and can be used in the kit and methods of the invention for treating diabetic and other sores or ulcers.

### Example 2

### Preparation of Pomegranate-Seeds Oil Extract

Pomegranate fruits are generally described as nearly round, 2½ to 5 in. wide, crowned at the base by the prominent calyx. The tough, leathery skin or rind is typically yellow overlaid with light or deep pink or rich red. The interior is separated by membranous walls and white, spongy, bitter tissue into compartments packed with sacs filled with sweetly acid, juicy, red, pink or whitish pulp or aril. In each sac there is one angular, soft or hard seed.

In order to prepare the seed extract, the below described procedure is followed:

1. Separate the sacs from the peel (all pomegranate species are suitable).
2. Squeeze the sacs to obtain pomegranate juice.
3. Ferment the sacs (what is left from it with the seeds) together with natural yeast, at 28° C. for a suitable period of time while mixing at suitable intervals.
4. Wash the resulting material with clean water while vortexing, which causes the seeds to brush against each other, a process that removes their outer membranes, which float and can be readily removed.
5. Dry the clean seeds.
6. Transfer the dry seeds to a vibrator for removal of any remaining peel, following which dry and clean seeds are separated from the dry and clean pomegranate peel (which may be used for preparing pomegranate peel powder).
7. Press the dry seeds to obtain oil; and optionally
8. Filter the oil to obtain clear oil.

The pomegranate oil obtained by this process was clear, of bright yellow color, and had a lightly sweet odor. The oil contained mostly unsaturated fatty acids, with linolenic acid being predominant (about 93%), and the level of saturated fatty acids was about 7%. The oil also contained phytosterols, mostly campesterol and β-sitosterol as well as vitamin E, mostly α-tocopherol. Viscosity was 4.22 cp.

The composition of a representative batch is described in Table 1 below, but variations are possible.

TABLE 1
Composition and properties of pomegranate oil obtained by
the method of the invention

| | Units/Method of | |
|---|---|---|
| | analysis | Value |
| Acid value | mg KOH/g | 0.28 |
| Peroxide Value | meq/Kg | 5.58 |
| Typical Properties | | |
| Iodine Value | g/100 g | ~236.1 |
| Refractive Index@40 cd | n | 1.5145 |
| Saponification Value | mg KOH/g | 191.5 |
| Unsaponifiable matter | % | 1.05 |
| Viscosity | cp | 4.22 |
| Moisture | % | 0.47 |
| Fatty Acid Composition | | |
| C16:0 | GLC | 3.37% |
| C18:0 | GLC | 2.4% |
| C18:1 | GLC | 7.52% |
| C18:2 | GLC | 5.84% |
| C18:3 | GLC | 79.12% |
| C20:0 | GLC | 0.47% |
| C20:1 | GLC | 0.85% |
| C22:0 | GLC | 0.083% |
| C24:0 | GLC | 0.046% |
| Others | GLC | 0.3% |
| Total Saturated | | 6.49% |
| Unsaturated | | |
| Mono | | 8.45% |
| Di unsaturated | | 5.85% |
| Tri unsaturated | | 79.12% |
| Total Unsaturated | | 93.44% |
| Saturated/Unsaturated | | 0.069 |
| Sterol Composition | GLC | |
| Cholesterol | mg/kg | 0.1 |
| Cholestanol | mg/kg | 0.3 |
| Brassicasterol | mg/kg | 0.0 |
| 24-methylene-cholesterol | mg/kg | 0.5 |
| Campesterol | mg/kg | 424.6 |
| Campestanol | mg/kg | 3.6 |
| Stigmasterol | mg/kg | 182.2 |
| Delta-7-capesterol | mg/kg | 11.4 |
| Delta-5,23-stigmastadienol | mg/kg | 10.3 |
| Celerosterol | mg/kg | 45.3 |
| Beta-sitosterol | mg/kg | 3726.8 |
| Sitostanol | mg/kg | 24.1 |
| Delta-5-avenasterol | mg/kg | 116.8 |
| Delta-5-24-stigmastadienol | mg/kg | 20.5 |
| Delta-stigmatenol | mg/kg | 27.4 |
| Delta-7-avenasterol | mg/kg | 49.2 |
| Total | mg/kg | 4643.5 |
| Vitamin E content | HPLC | |
| Alpha tocopherol | | 2013.4 |
| Beta tocopherol | | — |
| Gamma tocopherol | | 137.5 |
| Delta tocopherol | | 65.1 |
| Total | | 2216.0 |

The clear oil obtained in this Example may be packed in pierceable gelatin capsules for topical use in accordance with the invention.

### Example 3

### Preparation of Pomegranate Peel Powder

1. Whole pomegranates are crushed.
2. The dry material (i.e. peel and seeds) is introduced into a fermentation vessel together with natural yeast, and the mixture is fermented at 28° C. for a suitable period of time, mixing at suitable intervals.
3. The resulting material is washed with clean water for removal of the seeds outer membrane.
4. The solid material obtained is dried 42° C. for a suitable period of time, in a dry room.
5. The dry material obtained in step 4 is introduced into a vibrator for separating the peel and the seeds; and
6. The dry peel is ground to a powder.

The obtained powder possesses therapeutic properties and is used as a component of the therapeutic kit of the invention.

### Example 4

### Treatment of Diabetic Sores

1. Sores and their surroundings are washed thoroughly with clean water, and/or with a dilute hydrogen peroxide solution (or any other disinfectant solution suitable for cleaning sores), and are then dried, preferably with absorbing wipers;
2. Optionally, the flowers and peel extract is sprayed onto the sores;
3. The pomegranate oil is sprayed on the sore;
4. The pomegranate peel powder is then applied over the sore (on top of the oil);
5. When the sores are on the sole, they are preferably bandaged; if in other parts of the foot/leg or body, the sores are preferably left un-bandaged, exposed to fresh air;
6. Steps (1)-(4) are repeated at least twice a clay.

Experiments conducted by the present inventor showed almost immediate relief, and healing within 30-60 days.

### Clinical Studies

### Case 1:

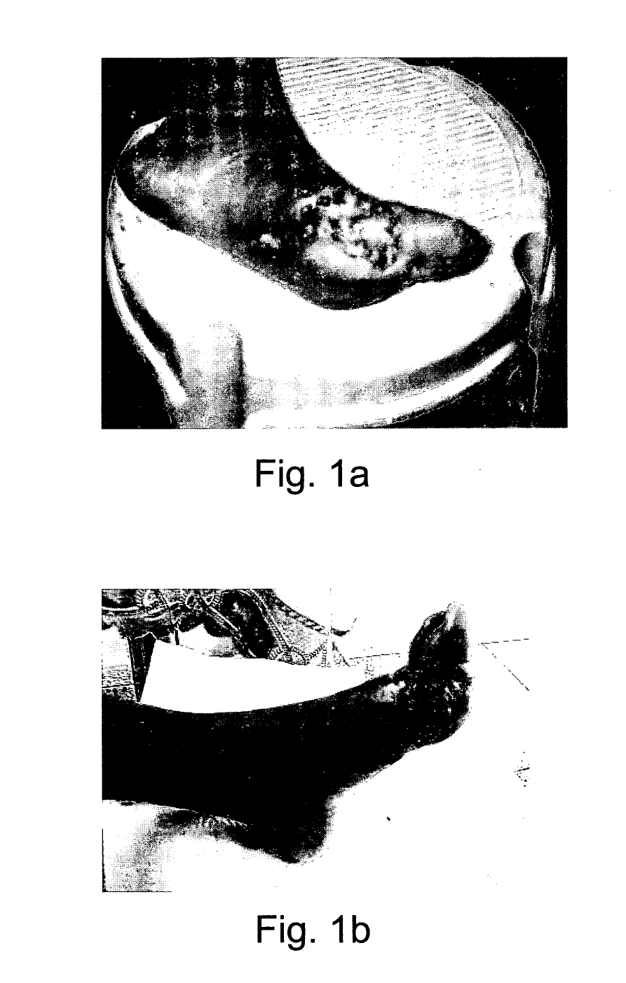

S. A. is a 62 years old diabetic patient, suffering from diabetes for 7 years. He suffered from severe diabetic sores, and his foot was to be amputated (see FIG. 1a). Following 30 days of treatment in accordance with the method of the invention, a significant improvement was observed (see FIG. 1b). Treatment is to last about 45 days, when complete healing is expected.

### Case 2:

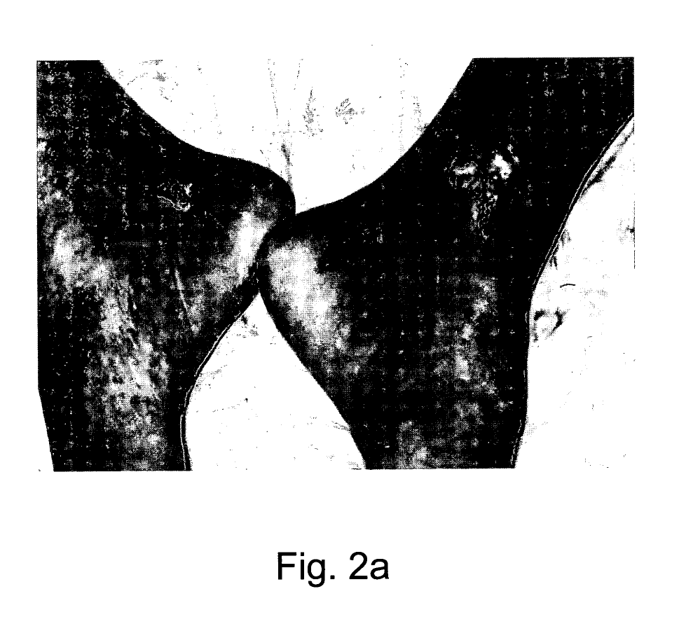

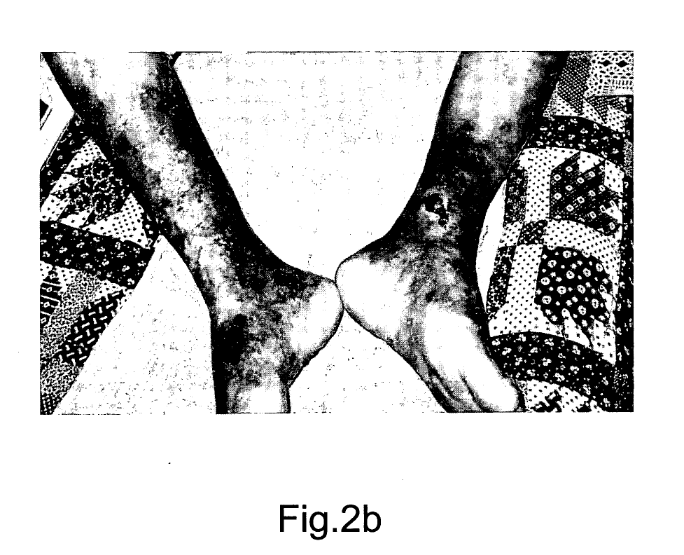

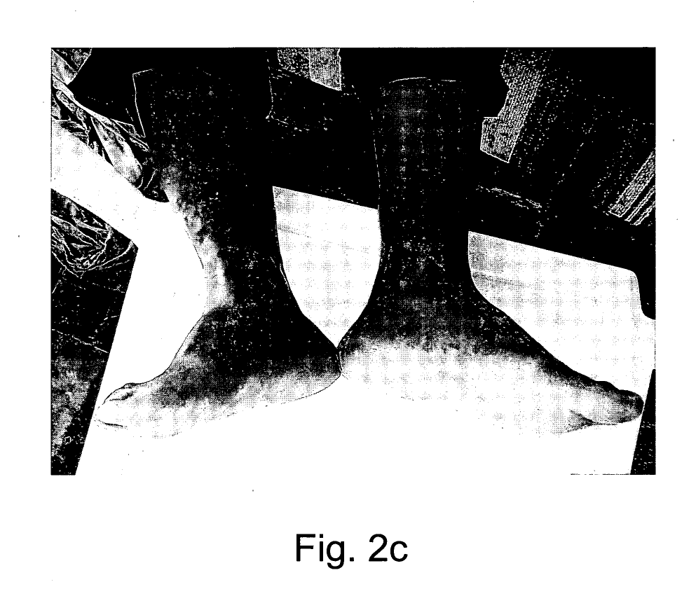

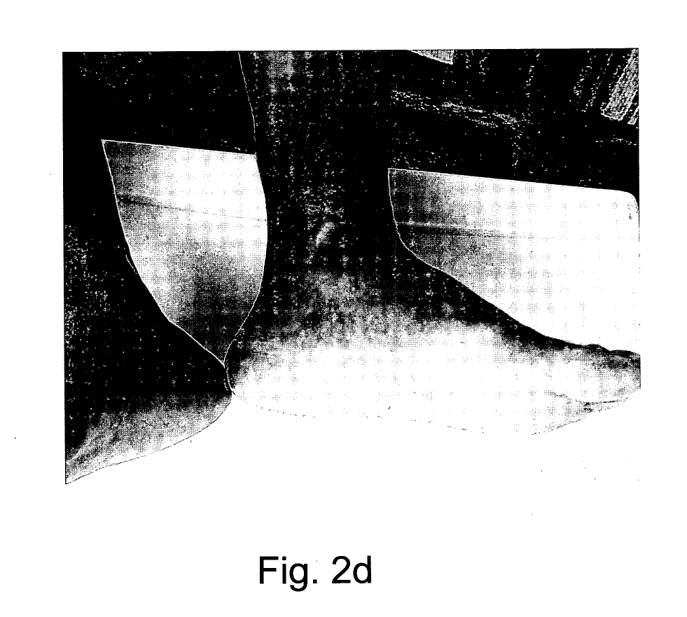

The patient A. Z., male, 57 yeas old, suffering from hyperlipidemia and diabetes for two years, and presenting diabetes-related leg sores. The patient was treated with conventional drugs used or the treatment of diabetes, such as Glucophage® (Brystol-Myers Squibb), Metformin, Glucomin (generic metformin) and Gluben® (glibenclamide, Dexcel). The patient was released from the hospital without improvement, and for continued treatment at home with Vaseline®. The patient suffered from swollen legs, with redness and open purulent sores. The sores were painful to such a degree that the patient needed treatment with morphine for easing the pain. The patient was referred to the present inventor and started the treatment which consisted of disinfecting the sores and applying flower extract, oil and powder. During one month, the sores were treated twice daily, in the morning and in the evening, followed by once daily during the next month. The improvement in the sores allowed the patient to get back to work. FIGS. 2a-2d show photographs of the sores at the beginning, during and at the end of the treatment.

### Case 3:

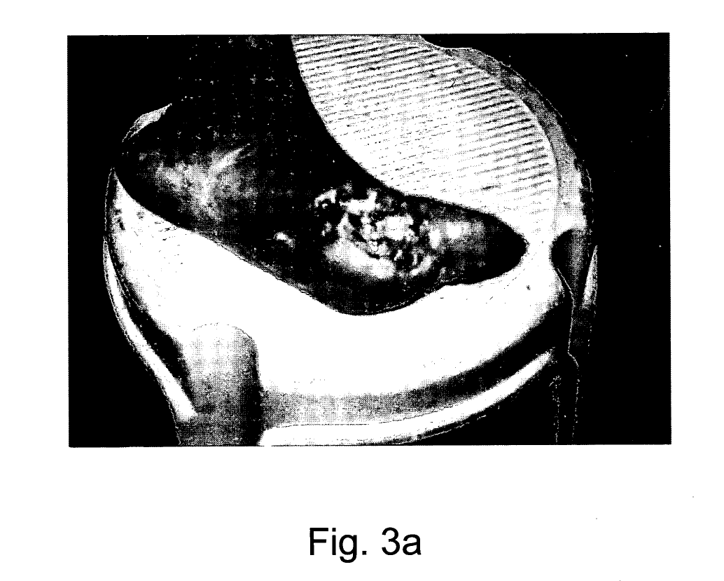

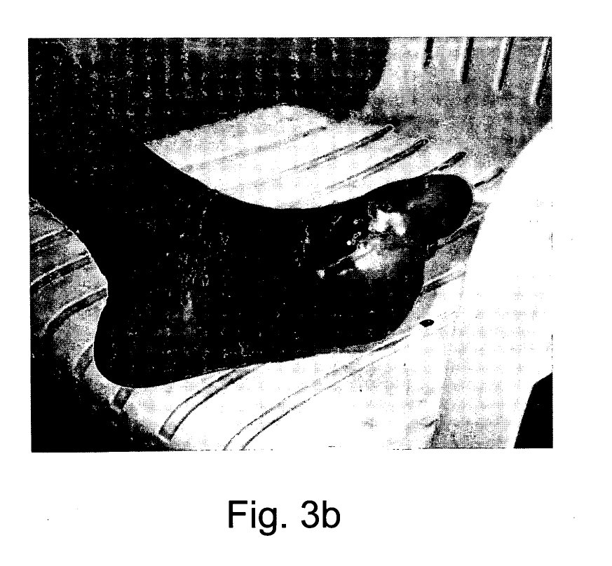

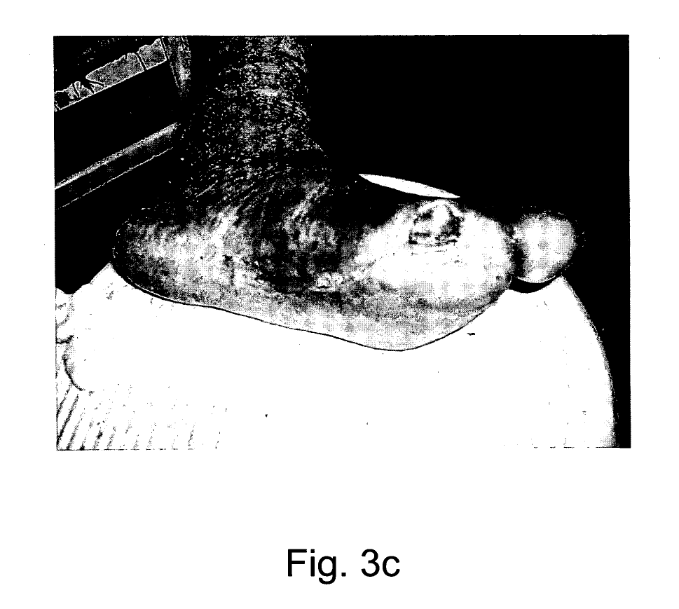

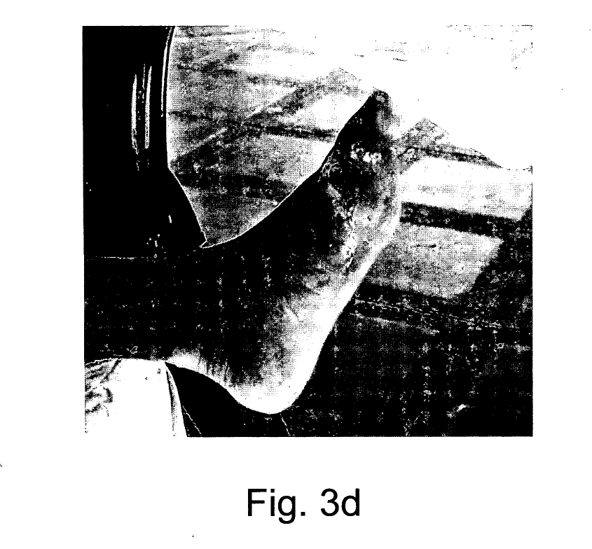

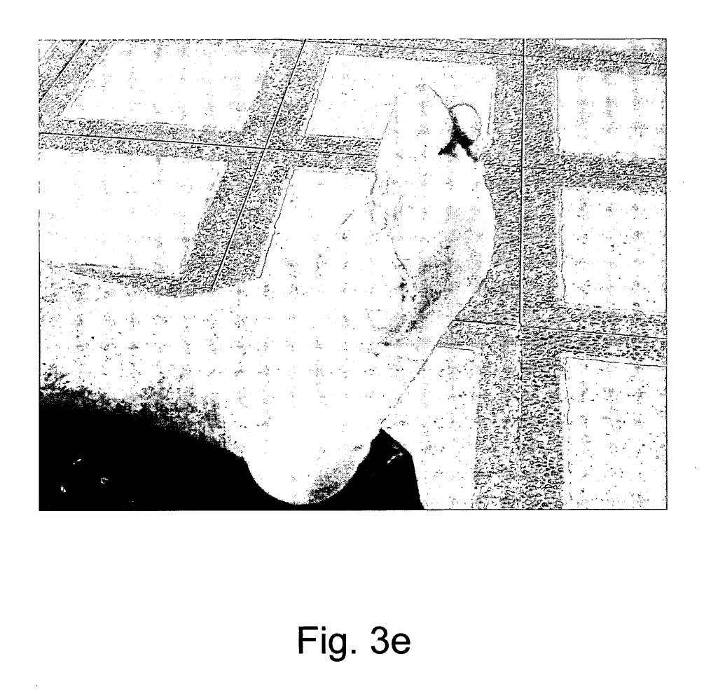

The patient S. A., male, 68 yeas old, suffering from high blood pressure, hyperlipidemia and diabetes for twenty years, and presenting diabetes-related leg sores. The patient was treated with conventional drugs used or the treatment of diabetes, such as Glucophage® (Brystol-Myers Squibb), Metformin, Glucomin (generic metformin) and Gluben® (glibenclamide, Dexcel). The patient was treated in one hospital with no improvement, where it was suggested that part of the leg should be amputated. The patient refused the amputation and referred to a second hospital, from which he was released without improvement. The patient was referred to the present inventor and started the treatment which consisted of disinfecting the sores and applying flower extract, oil and powder. During two months, the sores were treated twice daily, in the morning and in the evening, followed by once daily during the next month-and-a-half, until improvement which allowed the patient to walk and even drive by himself. FIGS. 3a-3e show photographs of the sores at the beginning, during and at the end of the treatment.

While some embodiments of the invention have been described by way of illustration, it will be apparent that the invention can be put into practice with many modifications, variations and adaptations, and with the use of numerous equivalents or alternative solutions that are within the scope of persons skilled in the art, without departing from the spirit of the invention or exceeding the scope of the claims.

1. A process for the preparation of pomegranate seeds oil extract comprising the following steps:
a) providing isolated pomegranate seeds;
b) cold-pressing the seeds to obtain an oil; and optionally
c) pressure-filtering the resulting oil, to obtain clear oil.

2. The process of claim 1, wherein said isolated pomegranate seeds are isolated from the fermentation product of pomegranate sacs fermented with natural yeast.

3. A process for the preparation of pomegranate-flower extract, comprising the following steps:
a) immersing pomegranate inflorescence in 70% alcohol at room temperature for a suitable period of time, while mixing at suitable intervals;
b) steaming and thickening the mixture obtained in step (a) until the alcohol has been removed to give a pomegranate flower extract.

4. A process for the preparation of pomegranate peel extract from pomegranates, comprising the following steps:
a) immersing pomegranate inflorescence in 70% alcohol at room temperature for a suitable period of time, while mixing at suitable intervals;
b) steaming and thickening the mixture obtained in step (a) until the alcohol has been removed to give a pomegranate peel extract.

5. A process for the preparation of pomegranate inflorescence/peel extract, comprising mixing the inflorescence extract obtained by the process of claim 3 with the pomegranate peel extract obtained by the process of claim 4 at a 1:1 ratio (v/v).

6. A process for the preparation of pomegranate peel powder from pomegranates, comprising the steps of:
a) crushing whole pomegranate;
b) fermenting the dry material (i.e. peel and seeds) obtained in step (a) together with natural yeast at 28° C. for a suitable period of time, while mixing at suitable intervals;
c) washing the resulting material with water to remove the seed membrane;
d) drying the material obtained in step (c) at 42° C.;
e) transferring the dry material obtained in step (d) to a vibrator for the separation of the peel from the seeds; and
f) grinding the dry peel to powder.

7. A pomegranate seed oil extract obtainable by the process of claim 1 or claim 2.

8. A pomegranate flower extract obtainable by the process of claim 3.

9. A pomegranate peel extract obtainable by the process of claim 4.

10. A pomegranate inflorescence/peel extract obtainable by the process of claim 5.

11. A pomegranate peel powder obtainable by the process of claim 5.

12. A medical composition for the treatment of skin sores, comprising as active ingredient at least one of pomegranate seed oil and pomegranate flower extract or pomegranate peel extract.

13. The composition of claim 12, wherein said pomegranate seed oil extract is obtainable by the process of claim 1 or 2, and/or said pomegranate flower+peel extract is obtainable by the process of claim 5 and/or said pomegranate peel powder is obtainable by the process of claim 6.

14. The composition of claim 12 or claim 13, wherein said skin sores are diabetic sores, pressure sores, ulcers or acne vulgaris.

15. A kit for the treatment of skin sores comprising:
a) pomegranate flower+peel extract;
b) pomegranate oil extract;
c) pomegranate peel powder;
d) means for applying the said pomegranate flower+peel extract, pomegranate oil extract and/or pomegranate peel powder to said skin sores;
e) optionally bandages; and
f) instructions for use.

16. The kit of claim 15, wherein said pomegranate seed oil extract is obtainable by the process of claim 1 or 2, and/or said pomegranate flower+peel extract is obtainable by the process of claim 5, and/or said pomegranate peel powder is obtainable by the process of claim 6.

17. The kit of any one of claim 15 or 16, wherein said pomegranate flower+peel extract is preferably contained in an atomizer.

18. The kit of any one of claims 15 to 17, wherein said skin sores are any of diabetic sores, pressure sores, ulcers and acne vulgaris.

19. A method of treating skin sores, comprising the following steps:
a) washing the sore thoroughly, and drying it and its surroundings;
b) applying to the sore pomegranate flowers+peel extract;
c) applying to the sores pomegranate oil extract;
d) powdering the sores with pomegranate peel powder;
e) optionally bandaging the sore; and
f) repeating steps (a)-(d) twice a day.

20. The method of claim 19, wherein said pomegranate seed oil extract is obtainable by the process of claim 1 or 2, and/or said pomegranate flower+peel extract is obtainable by the process of claim 5, and/or said pomegranate peel powder is obtainable by the process of claim 6.

21. The method of claim 19 or claim 20, wherein said skin sores are diabetic sores, pressure sores, ulcers or acne vulgaris.

22. The method of any one of claims 19 to 21, wherein pomegranate flowers+seed extract or pomegranate oil extract are applied by spraying.

23. A method of preventing skin sores in a patient in need, comprising the following steps:
a) applying to the skin of said patient pomegranate oil extract;
b) powdering with pomegranate peel powder; and optionally
c) repeating steps (a)-(d) twice a day.

24. The method of claim 23, wherein said pomegranate seed oil extract is obtainable by the process of claim 1 or 2, and/or said pomegranate flower+peel extract is obtainable by the process of claim 5, and/or said pomegranate peel powder is obtainable by the process of claim 6.

25. The method of any one of claim 23 or 24, wherein said skin sores are any of diabetic sores, pressure sores, ulcers or acne vulgaris.

26. The method of any one of claims 23-25, wherein said pomegranate oil extract is applied by spraying.
